(19) United States
(12) Patent Application Publication
(10) Pub. No.: US 2026/0096832 A1
(43) Pub. Date: Apr. 9, 2026

(54) DEVICES AND METHODS FOR CREATING CAVITIES IN SPINE SURGERY

(71) Applicant: Wei HSIUNG, New Taipei City (TW)

(72) Inventor: Wei HSIUNG, New Taipei City (TW)

(21) Appl. No.: 18/906,161

(22) Filed: Oct. 3, 2024

(51) Int. Cl. A61B 17/3209; A61B 17/32

(52) U.S. Cl. CPC A61B 17/3209; A61B 17/320016

(57) ABSTRACT

Disclosed herein is a device for use in a spine surgery. The device includes: a first member having a distal end and a proximal end and includes a first lumen extending through the axial length of the first member and has a proximal opining for introducing a first fluid therethrough; a second member having a distal end and a proximal end and includes a second lumen sized to accommodate the first member therein except the distal and proximal ends thereof, a third lumen having a distal opening and is parallel to the second lumen, and a proximal inlet connected to the third lumen for introducing a second fluid therethrough; and an expandable structure coupled to the distal opening of the third lumen, wherein the first member is made of a rigid material, while the second member is made of a flexible material; and the second fluid is to expand the expandable structure. Also disclosed herein is a method for treating a spine disease by use of the device disclosed herein.

### BACKGROUND OF THE INVENTION

### 1. Field of the Invention

The present disclosure relates to a device for use in a spine surgery. More particularly, the disclosure invention relates to a device for creating a cavity within a treatment site of a subject in need of a spine surgery.

### 2. Description of Related Art

A number of medical conditions (e.g., a bone disease) require creating a cavity within a live body for receiving surgical tools therein to repair and/or remove damage tissue. Traditionally, an opening is made from the skin and a distractor is commonly inserted to push away the soft tissue around the vertebral disc thereby creating the needed cavity. With the development of endoscopic instruments, minimally invasive surgery, particularly, unilateral biportal endoscopic (UBE) spine surgery, that requires making only 1 or 2 very small holes on the patient's body to reach the target tissue inside becomes the prevalent technique. However, as the hole in minimally invasive surgery is too small to use the distractor, physician elects to use bipolar instrument to ablate the soft tissue around the target tissue to create cavity for surgery. However, the soft tissue should be push away, just like in the conventional surgery, instead of being ablated in UBE spine surgery.

Accordingly, there exists in the related art a need of a specialized instrument for use with UBE surgery to create a cavity for the surgery.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a device for use in a spine surgery (e.g., UBE surgery). The device comprises a first member having a distal end and a proximal end that shaped as a handle; a second member having a distal end and a proximal end; and an expandable structure coupled to the second member, wherein the second member is disposed around and along the axial length of the first member.

According to embodiments of the present disclosure, the first member comprises a first lumen extending through the axial length of the first member and has a proximal opining for introducing a first fluid therethrough.

According to embodiments of the present disclosure, the second member comprises a second lumen sized to accommodate the first member therein except the distal and proximal ends thereof; a third lumen having a distal opening and is parallel to the second lumen; and a proximal inlet connected to the third lumen for introducing a second fluid therethrough.

According to embodiments of the present disclosure, the expandable structure is disposed at and around the distal end of the second member and is coupled to the distal opening of the third lumen. Further, the expandable structure is expanded by the second fluid introduced through the proximal inlet of the third lumen of the second member.

According to embodiments of the present disclosure, the first member is made of a rigid material, such as stainless steel; while the second member is made of a flexible material selected from the group consisting of teflon, polyethylene (PE), polypropylene (PP), and silicates.

According to embodiments of the present disclosure, the distal end of the first member is in the form of a curette or a tip.

According to embodiments of the present disclosure, the first fluid is water, saline, an anti-coagulant, or a hydrogel; and the second fluid is water or saline.

According to embodiments of the present disclosure, the expanded structure is a balloon.

According to embodiments of the present disclosure, the expandable structure is about 2-3 mm from the distal end of the first member after expansion.

In a further aspect, the present disclosure is directed to a method for treating a spine disease of a subject via use of any one of the devices of the present disclosure. The method includes steps of:
(a) making first and second skin incisions above a target site of the subject;
(b) inserting the device of any one of claims 1 to 6 and an endoscope respectively through the first and second skin incisions until they reach the target site in the spine;
(c) making a void in the target site by the distal end of the first member of the device;
(d) introducing water through the proximal inlet of the second member to expand the expandable structure thereby further expanding the void;
(e) introducing an anti-coagulant and/or a hydrogel from the proximal opening of the first lumen to the further expanded void of step (d) thereby forming a cavity for receiving a surgical instrument;
(f) withdrawing the water from the proximal inlet of the second member to collapse the expandable structure;
(g) inserting a surgical instrument to the cavity; and
(h) performing a surgery with the surgical instrument in the cavity to treat the spine disease of the subject.

According to embodiments of the present disclosure, in step (a), the target site is an intervertebral disc.

Examples of the spine disease suitable for treating by the present method include, but are not limited to, herniation of intervertebral disc (HIVD), spine canal stenosis, foraminal stenosis, ossification of the ligament flavum, low-grade spondylolisthesis, and adjacent segment degeneration.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1A is an exploded view of a device 100 in accordance with one embodiment of the present disclosure;

FIG. 1B depicts the device 100 constructed by the components of FIG. 1A for use in a spine surgery; and

FIG. 1C is a cross-sectional view of the second member 120 of FIG. 1A taken along the line L-L′ in accordance with one embodiment of the present disclosure.

In accordance with common practice, the various described features/elements are not drawn to scale but instead are drawn to best illustrate specific features/elements relevant to the present invention. Also, like reference numerals and designations in the various drawings are used to indicate like elements/parts.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definition

For convenience, certain terms employed in the specification, examples and appended claims are collected here. Unless defined otherwise, all technical and scientific terms used herein have the same meaning as commonly understood by one of the ordinary skilled in the art to which this invention belongs.

Throughout the present disclosure, “distal” is understood to mean away from the practitioner holding the present device towards the site the present device is applied. “proximal” is understood to mean towards the practitioner and away from the site the device is applied.

The singular forms “a”, “and”, and “the” are used herein to include plural referents unless the context clearly dictates otherwise.

### 2. The Present Device

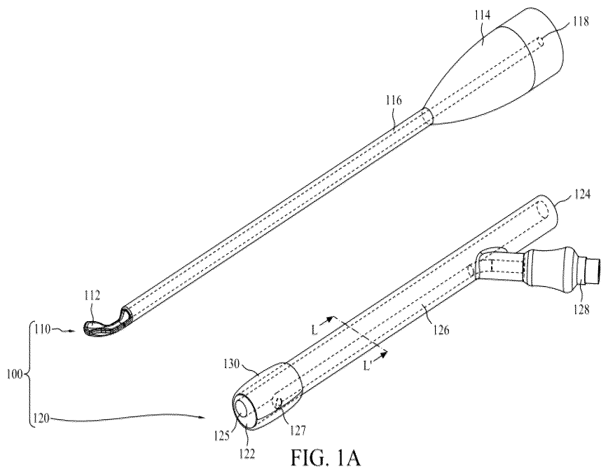

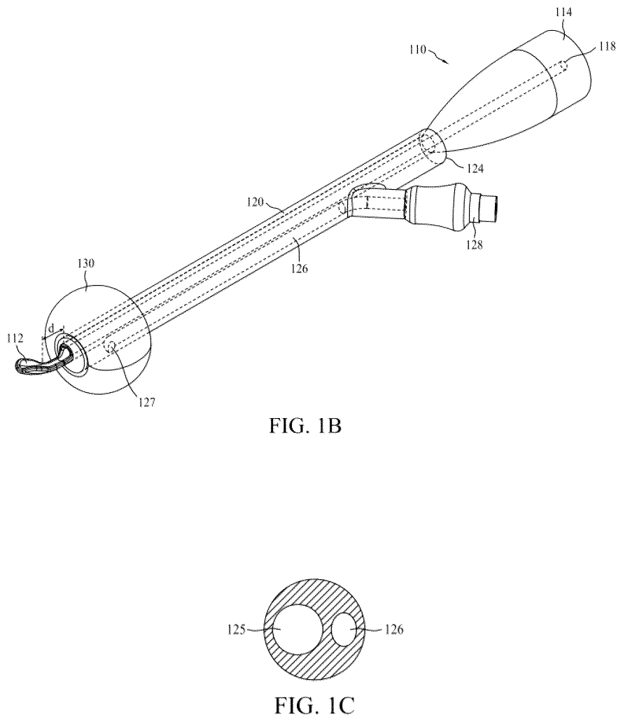

References are made to FIGS. 1A to 1C, in which FIG. 1A is an exploded view of a device 100 in accordance with one embodiment of the present disclosure; FIG. 1B is a schematic diagram depicting the device 100 constructed by the components of FIG. 1A for use in a spine surgery, such as UBE spine surgery; and FIG. 1C is a cross-sectional view of the second member 120 of FIG. 1A taken along the line L-L′. Specifically, the device 100 comprises in its structure, a first member 110, a second member 120, and an expandable structure 130 coupled to the second member 120, in which the second member 120 is disposed around and along an axial length of the first member 110.

The first member 110 is generally a one-piece integrally formed hollow member with a shape similar to a conventional curette. Structurally, the first member 110 has a distal end 112 and a proximal end 114, and a first lumen 116 extending through the axial length of the first member 110 and has a proximal opining 118 for introducing a first fluid therethrough. According to embodiments of the present disclosure, the proximal end 114 of the first member 110 is shaped as a handle to be held by a single hand of a practitioner (e.g., a doctor) during surgery; while the distal end 112 of the first member 110 is shaped as a curette or a tip designed to create a cavity within a target site of a body, thus, either form of the distal end 112 is suitable for removing tissue (e.g., bone tissue) from the target site (e.g., a vertebra) thereby creating a void therein. Note that in FIG. 1A or 1B, the distal end 112 of the first member 110 is in the form of a curette. Alternatively, the distal end 112 may be in the form of a tip, which may further include a stopper just before the tip, in which the stopper serves as a baffle to prevent the bone tissue from being over removed during cavity creating.

The second member 120 is generally a bi-lumen tube with a distal end 122 and a proximal end 124, and comprises in its structure, a second lumen 125, a third lumen 126, and a proximal inlet 128 connected to the third lumen 126 for introducing a second fluid therethrough, and the second and third lumens 125, 126 share a common wall in the second member 120. The second lumen 125 extends through the axial length of the second member 120 and is sized to accommodate the longitudinal part the first member 110 therein, that is, the part of the first member 110 without the distal and proximal ends 112, 114 (FIG. 1B). In some alternative embodiments, the second lumen 125 is sized to accommodate the part of the first member 110 without the proximal end 114. The third lumen 126, which share a common wall with the second lumen 125, has a distal opening 127, in which the expandable structure 130 is removably carried thereby. Note that in FIG. 1A, the expandable structure 130 is in deflated state; while in FIG. 1B, the expandable structure 130 is in inflated state. According to embodiments of the present disclosure, the expandable structure 130 is disposed at and around the distal end 122 of the second member 120 and is coupled to the distal opening 127 of the third lumen 126. The expandable structure 130 (e.g., a ballon) may be expanded by the second fluid introduced through the proximal inlet 128. Specifically, a fluid (e.g., water) may enter from the proximal inlet 128 and flow through the distal opening 127 and accumulate within the expandable structure 130 thereby expanding the expandable structure 130 (e.g., an inflated balloon). The expanded structure 130 may push away neighboring tissues and further expand the void created by the curette 112.

According to preferred embodiments, an anti-coagulant and/or a hydrogel may be introduced from the proximal opening 118 of the first lumen 116 to the expanded void, thereby forming the desired cavity, where surgical tools and/or medicament may work and/or applied therein to remove and/or repair any diseased tissues.

Additionally, or optionally, with the desired cavity being formed, the expanded structure 130 may then be contracted and removed through the third lumen 126. To this purpose, water in the expanded structure 130 is withdrawn (e.g., via a syringe or a pump) and taken out of the third lumen 126 through the proximal inlet 128 thereby collapsing the expanded structure 130, allowing it to be removed from the second member 120.

### 3. Methods for Treating a Spine Disease

Also encompassed herein is a method for treating a spine disease. Specifically, a method for creating a cavity in a subject in need of a spine surgery to treat a spine disease by use of the device of the present disclosure.

To create a cavity suitable for performing a spine surgery for the treatment of a spine disease, two skin incisions are made above a target site (e.g., an intervertebral disc) of the subject. Then, the present device 100 and an endoscope are respectively inserted through the two skin incisions until they reach the target site in the spine, in which the endoscope may provide the practitioner a better view within the target site so as to help positioning and/or guiding the operation of the present device 100 in the target site.

Once the device 100 is positioned in the target site (i.e., the targeted intervertebral disc), the first and second members 110, 120, specifically, the distal end 112, and the expanded structure 130 of the device 100 may work together to create a cavity suitable for performing a surgery and/or applying a treatment therein to treat the spine disease. First, the practitioner may distally move or angularly rotate the first member 110 with the aid of the handle 114, which in turn results in distally moving or angularly rotating the distal end 112 of the first member 110 to scrap off neighboring bone tissues and forms a void space in the bone.

Once the void is formed, water is injected from the proximal inlet 128 of the third lumen 126 of the second member 120 to expand the expandable structure 130 carried at the end of the distal opening 127 of the third lumen 126. According to preferred embodiments of the present disclosure, the expandable structure 130 is distanced (d) from the distal end 112 of the first member 110 for about 2-3 mm, such as 2 mm or 3 mm after expansion (FIG. 1B). The inflated expandable structure 130 (e.g., a ballon filled with water) may push away neighboring tissues further away thereby expanding the void previously created by the distal end 112 of the first member 110. More preferably, an anti-coagulant and/or a hydrogel is introduced from the proximal opening 118 of the first lumen 116 to the expanded void to further strengthen the structure therein, thereby forming the desired cavity strong enough to allow surgical tools to work therein subsequently to remove and/or repair any diseased tissues.

With the formation of the desired cavity, the inflated expandable structure 130 (i.e., the inflated balloon) may be contracted and removed through the third lumen 126. To this purpose, water in the expanded structure 130 is withdrawn (e.g., via a syringe or a pump) and taken out of the third lumen 126 through the proximal inlet 128 thereby collapsing the expandable structure 130, allowing it to be removed from the third lumen 126. After which, surgical tools and/or suitable medicament (e.g., bone cement) may be introduced into the cavity. Surgery (e.g., laminotomy) may then be performed within the cavity to treat the spine disease by removing damage tissue therein. After surgery, surgical tools are removed by backwardly retracting them through the same route.

According to embodiments of the present disclosure, the present device and method are suitable for creating cavity in a target site of a subject suffering from a spine disease. Examples of spine disease include, but are not limited to, herniation of intervertebral disc (HIVD), spine canal stenosis, foraminal stenosis, ossification of the ligament flavum, low-grade spondylolisthesis, adjacent segment degeneration, and the like.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A device for use in a spine surgery comprising:
a first member having a distal end and a proximal end that shaped as a handle and comprises:
a first lumen extending through the axial length of the first member and has a proximal opining for introducing a first fluid therethrough;
a second member having a distal end and a proximal end and comprises:
a second lumen sized to accommodate the first member therein except the distal end or the distal and proximal ends thereof;
a third lumen having a distal opening and is parallel to the second lumen; and
a proximal inlet connected to the third lumen for introducing a second fluid therethrough; and
an expandable structure disposed at and around the distal end of the second member and is coupled to the distal opening of the third lumen;
wherein,
the first member is made of a rigid material, while the second member is made of a flexible material; and
the second fluid is to expand the expandable structure.

2. The device of claim 1, wherein the distal end of the first member is in the form of a curette or a tip.

3. The device of claim 1, wherein the first fluid is water, saline, an anti-coagulant, or a hydrogel; and the second fluid is water or saline.

4. The device of claim 1, wherein the expandable structure is a ballon.

5. The device of claim 4, wherein the ballon is about 2-3 mm from the distal end of the first member after expansion.

6. The device of claim 1, wherein the first member is made of stainless steel; and the second member is made of teflon, polyethylene (PE), polypropylene (PP), or silicates.

7. A method for treating a spine disease of a subject comprising:
(a) making first and second skin incisions above a target site of the subject;
(b) inserting the device of claim 1 and an endoscope respectively through the first and second skin incisions until they reach the target site in the spine;
(c) making a void in the target site by the distal end of the first member of the device;
(d) introducing water through the proximal inlet of the second member to expand the expandable structure thereby further expanding the void;
(e) introducing an anti-coagulant and/or a hydrogel from the proximal opening of the first lumen to the further expanded void of step (d) thereby forming a cavity for receiving a surgical instrument;
(f) withdrawing the water from the proximal inlet of the second member to collapse the expandable structure;
(g) inserting a surgical instrument to the cavity; and
(h) performing a surgery with the surgical instrument in the cavity to treat the spine disease of the subject.

8. The method of claim 6, wherein in step (a), the target site is an intervertebral disc.

9. The method of claim 6, wherein the spine disease is herniation of intervertebral disc (HIVD), spine canal stenosis, foraminal stenosis, ossification of the ligament flavum, low-grade spondylolisthesis, or adjacent segment degeneration.
